(19) United States
(12) Patent Application Publication
(10) Pub. No.: US 2012/0302841 A1
(43) Pub. Date: Nov. 29, 2012

(54) BIOMETRIC HEALTHCARE DATA SYSTEM

(75) Inventors: Timothy W. Coressel, Arvada, CO (US); Rudolph J. Liedtke, Grosse Point Park, MI (US)

(73) Assignee: TELSANO HEALTH, Aurora, CO (US)

(21) Appl. No.: 13/478,213

(22) Filed: May 23, 2012

Related U.S. Application Data
(60) Provisional application No. 61/489,296, filed on May 24, 2011.

(51) Int. Cl. A61B 5/0205; A61B 5/1455; A61B 5/107; G01G 23/38; G06F 15/16; A61B 5/053

(52) U.S. Cl. 600/301; 709/217

(57) ABSTRACT

A biometric healthcare data system has biometric data collection stations and a network management system. Each station has several biometric data measurement devices including a bioelectrical impedance analysis system. Each station has a gateway that connects to the devices and that connects the station through the internet to the network management system. The network management system has a communications manager that tracks the stations, a content manager that provides content to the stations and a data manager that retrieves and stores data from the data collection stations.

This application claims the benefit under 35 U.S.C. §119(e) of the U.S. provisional patent application No. 61/489,296 filed May 24, 2011.

### TECHNICAL FIELD

The present invention relates to biometric data and more particularly to a system for collection and management of biometric data.

### BACKGROUND ART

Many standalone biometric measurement devices for healthcare are available. Examples of standalone biometric measurement devices include weighing scales, thermometers, pulse oximetry sensors, sphygmomanometers or blood pressure meters, height measuring devices, glucose meters and bioelectrical impedance analyzers. Measurements from these standalone devices are recorded by hand or are not recorded at all.

### DISCLOSURE OF THE INVENTION

A biometric healthcare data system includes one or more data collection stations and a network management system. Each data collection station includes a console and a plurality of biometric measurement devices connected to the console. The console has an input/output device, network communications interfaces, peripheral ports and a gateway that controls the station. The gateway includes multimedia input and output interfaces, a protocol converter, a user interface controller and a peripheral interface adapter. The network management system connects to the data collection stations through the internet and/or through the cellular network, and has a communications manager, a content manager and a data manager. The communications manager tracks the data collection stations. The content manager provides content to the data collection stations. The data manager retrieves and stores data from the data collection stations.

### BRIEF DESCRIPTION OF THE DRAWINGS

Details of this invention are described in connection with the accompanying drawings that bear similar reference numerals in which:

FIG. 1 is an illustrative view of a biometric healthcare data system embodying features of the present invention.

FIG. 2 is a front elevation view of a data collection station of the system of FIG. 1.

FIG. 3 is a side elevation view of the station of FIG. 2.

FIG. 4 is a top plan view of the station of FIG. 2.

FIG. 5 is a diagramic view of the circuit of the bioelectrical impedance analysis system of the station of FIG. 2.

FIG. 6 is a back elevation view of a hand grip of the console of FIG. 5.

FIG. 7 is a block diagram of a gateway for the station of FIG. 2.

FIG. 8 is a view of screen shot of the display on the console of the station of FIG. 2.

FIG. 9 is a block diagram of a network management system for the system of FIG. 1.

### DETAILED DESCRIPTION OF THE INVENTION

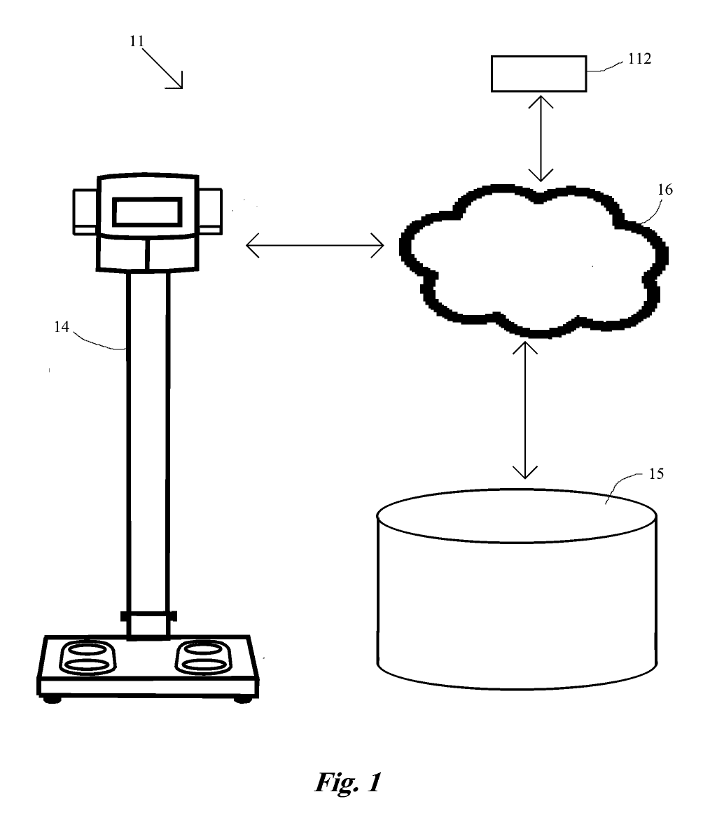

Referring to FIG. 1, a biometric healthcare data system 11, embodying features of the present invention, includes at least one and generally a plurality of data collection stations 14 connected to a network management system 15 preferably through the internet 16 and/or through the cellular network. Each data collection station 14 can function as a standalone unit or can be connected to the network management system 15.

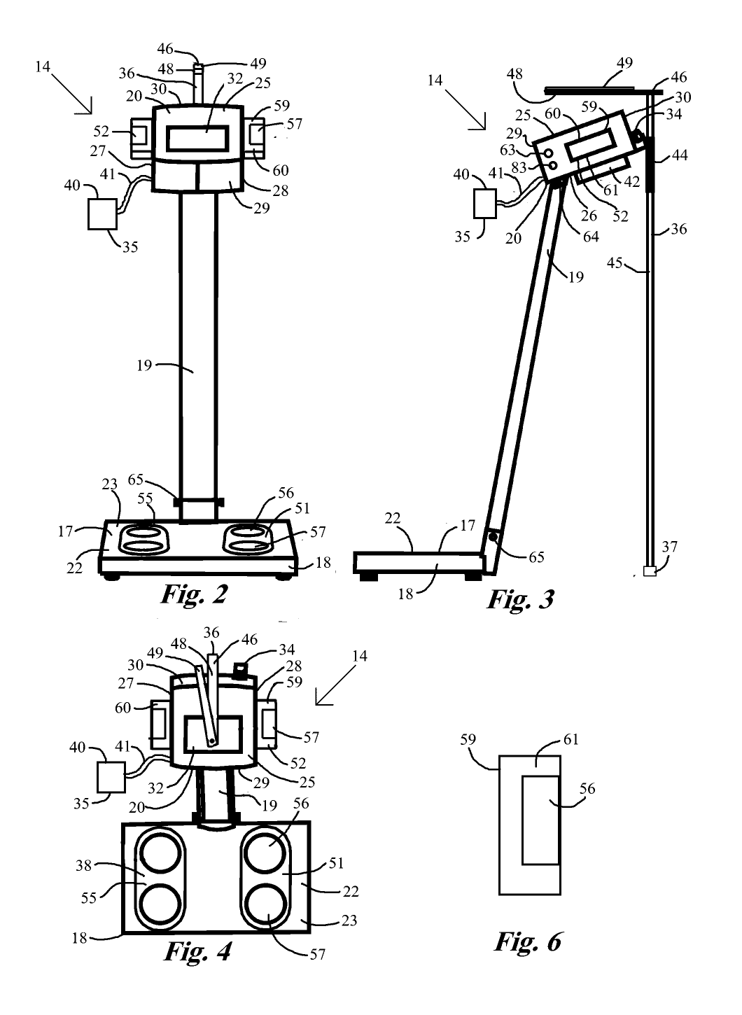

As shown in FIGS. 2 to 4, the data collection station 14 includes a plurality of biometric measurement devices 17. By way of example, and not as a limitation, the data collection station 14 can include a combination of blood pressure, heart rate, oxygen saturation, height, weight, temperature, half body and full body composition, glucose, and other measurement devices.

The data collection station 14 has a base 18, a console support 19 and a console 20. The base 18 has a low, flat box shape. A weighing scale 22 is incorporated into the base 18. The scale 22 has a flat, rectangular scale plate 23 that provides the top of the base 18. The console support 19 shown is an elongated rectangular tube that projects upwardly from the back of the base 18 and slants rearwardly. The console 20 is mounted on top of the console support 19. Preferably the length of the console support 19 is selected such that the console 20 is mounted above the scale plate 23 above the waist level of the average user.

The console 20 has a flattened box shape with an upper face 25, a spaced lower face 26, spaced left and right side walls 27 and 28 that extend from the upper face 25 to the lower face 26, and spaced front and back walls 29 and 30 that each extend from the left side wall 27 to the right side wall 28. The console 20 is tilted such that the upper face 25 faces upwardly and forwardly. A color touch screen display 32 is mounted on the upper face 25.

The biometric measurement devices 17 included in data collection station 14 shown are the scale 22, a pulse oximetry sensor 34, a blood pressure meter 35, a height measuring device 36, an ultrasonic pulse sensor 37 and a bioelectrical impedance analysis system 38. The pulse oximetry sensor 34 is mounted on the upper right on the back wall 30 of the console 20. The blood pressure meter 35 includes a cuff 40, a hose 41 that extends from the cuff 40 to the console 20 and a pump (not shown, located in the console 20). A cuff shelf 42, for stowing the cuff 40, is attached to the lower face 26 of the console.

The height measuring device 36 has a short tube 44 mounted vertically on the back wall 30 of the console 20, an elongated rod 45 that is sized to slide in the tube 44, and a top member 46. The top member 46 includes a first portion 48 that projects horizontally, forwardly from the top of rod 45 and a second portion 49 that is pivotally connected about a vertical axis to the first portion 48 opposite the rod 45. The second portion 49 is pivoted forwardly for height measurement and retracted rearwardly for storage. The ultrasonic pulse sensor 37 is mounted to the bottom of rod 45 and measures the distance from the bottom of the rod 45 to the floor to determine the height of a user.

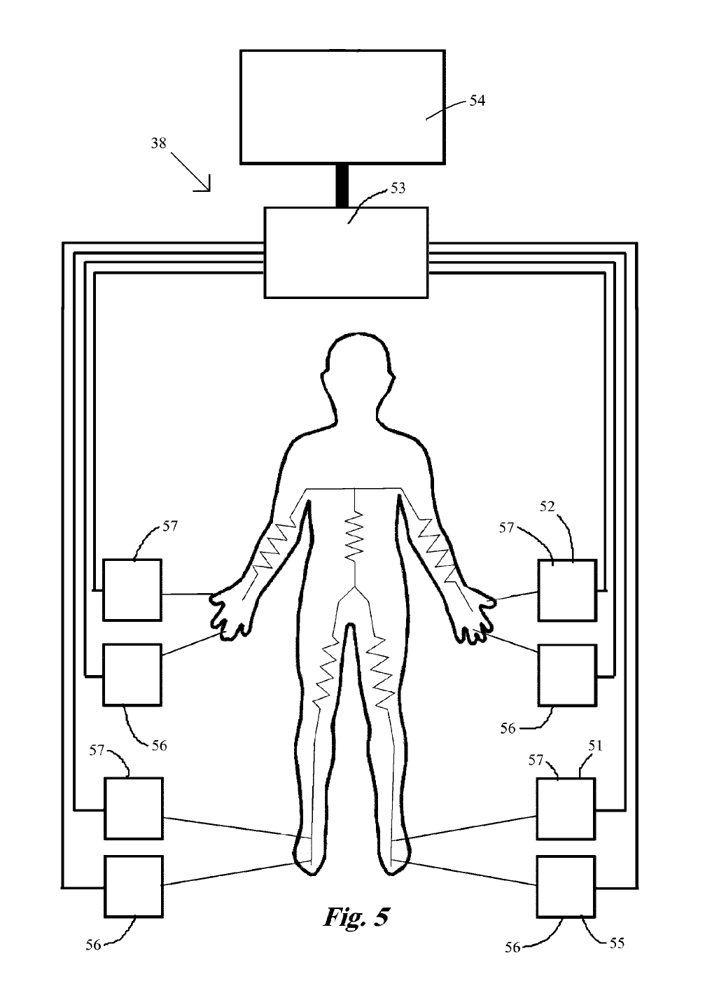

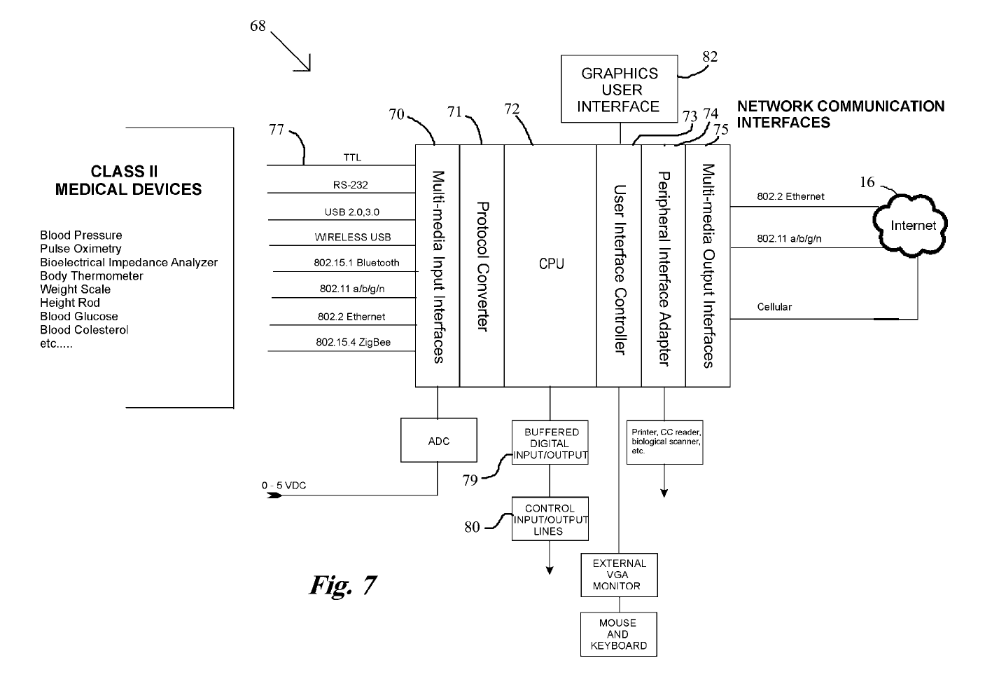

Referring to FIG. 5, the bioelectrical impedance analysis system 38 is a segmented bioelectrical impedance analysis system, and has two spaced electrode sets 55 of foot electrodes 51, two spaced electrode sets 55 of hand electrodes 52, a multiplexer 53, and a bioelectrical impedance analyzer 54 connected to the multiplexer 53. The multiplexer 53 and the bioelectrical impedance analyzer 54 are located inside the console 20. Each electrode set 55 includes a source electrode 56 connected to the multiplexer 53 and a spaced detector electrode 57 connected to the multiplexer 53. The bioelectrical impedance analysis system 38 measures the resistance and the reactance for each combination of source electrode 56 and detector electrode 57. The bioelectrical impedance analysis system 38 generates resistance and reactance values for the Right Body, Left Body, Lower Body, Upper Body, Right Arm, Left Arm, Right Leg, Left Leg, Right Torso, Left Torso, Transverse Right Arm to Left Leg, Transverse Left Arm to Right Leg, and Whole Body, resulting in twenty-six data points.

As shown in FIGS. 2 to 4, the foot electrodes 51 are positioned on the top of the scale plate 23 in a spaced arrangement. The foot electrodes 51 shown have the source electrode 56 located near the ball of the user's foot and the detector electrode 57 is located near the heel of the user's foot.

Each electrode set 55 of hand electrodes 52 includes a block 59 of non-conductive material. One block 59 is mounted on the left side wall 27 and one block 59 is mounted on the right side wall 28 of the console 20. Each block 59 has an upper surface 60 and a spaced, oppositely facing lower surface 61. The detector electrode 57 shown for each set of hand electrodes 52 is a conductive rectangular plate on the upper surface 60 of the block 59. FIG. 6 shows a source electrode 56 for the hand electrodes 52 as a conductive rectangular plate on the lower surface 61 of the block 59. The design of electrode sets 55 of hand electrodes 52 positions the fingertips of the hand on the source electrode 56 and the thumb on the detector electrode 57, and prevents the palm of the hand from contacting the hand electrodes, providing improved repeatability of the measurements.

An external set of electrodes can be connected to the multiplexer 53 of the bioelectrical impedance analysis system 38 for whole body bioelectrical impedance analysis. Referring again to FIGS. 2 to 4, a first external connector 63 is provided on the right side wall 28 of the console 20 for connecting an external set of electrodes.

The data collection station 14 is portable and can be disassembled. A first connection point 64 is provided between the top of the console support 19 and the console 20, for removal of the console 20 from the console support 19. A second connection point 65 is provided between the base 18 and the bottom of the console support 19, for removal of the console support 19 from the base 18.

The data collection station 14 includes a portable gateway 68. The gateway 68 has a multi-media input interface 70, a protocol converter 71, a central processing unit or CPU 72, a user interface controller 73, a peripheral interface adapter 74, and a multi-media output interface 75. The multi-media input interface 70 provides the physical, electrical and wireless connections between the gateway 68 and the biometric measurement devices 17. The multi-media input interface 70 is a means for interconnecting the biometric measurement devices 17 to the gateway 68. Several different wired and wireless communication media types 77 are supported by the multi-media input interface 70, including, but not limited to, TTL, RS-232, USB 2.0, 3.0, wireless USB, 802.15.1 Bluetooth, 802.15.5 Zigbee, 802.3 Ethernet, 802.11a/b/g/n wireless Ethernet, and analog to digital converter.

Each biometric measurement device 17 may transmit a standard or proprietary protocol and the biometric measurement devices 17 may transmit at different speeds. The protocol converter 71 is connected to the multi-media input interface 70 and is responsible for communications to and from each biometric measurement device 17. Data retrieved by the protocol converter 71 from each biometric measurement device 17 is passed by the CPU 72 to the user interface controller 73.

The CPU 72 is the brains behind the gateway 68 and provides the interconnectivity between the multi-media input interface 70, the protocol converter 71, the user interface controller 73, the peripheral interface adapter 74 and the multi-media output interface 75. The CPU 72 is connected to buffered digital input/output 79 which is connected to control input/output lines 80 to control or monitor external elements or devices. As an example, the CPU 72 can enable a relay which connects or disconnects an external set of electrodes to the bioelectrical impedance analysis system 38.

The user interface controller 73 is the input/output interface between the biometric measurement devices 17 and a user of the data collection station 14. The user interface controller 73 connects through a graphical user interface 82 to the touchscreen display 32, and to other user interfaces such as a keyboard, a mouse or an external monitor. The user interface controller 73 processes and passes commands from the user to the protocol converter 71 which then activates and collects data from the biometric measurement devices 17 connected to the multi-media input interface 70.

The peripheral interface adapter 74 interconnects external devices such as printers, credit card readers, biological input devices, bill acceptors, scanners and usb drives to the gateway 68. The multi-media output interface 75 includes the physical, electrical and wireless connections from the gateway 68 to the internet 16 and cellular networks, providing two-way communication between the gateway 68 and the internet 16 and cellular network. The multi-media output interface 75 supports several different communication media types 77 including 802.3 Ethernet, 802.11a/b/g/n wireless Ethernet, and cellular (i.e. CDMA, GSM). A second external connector 83, for wired Ethernet connection, is located on the console 20 adjacent to the first external connector 63.

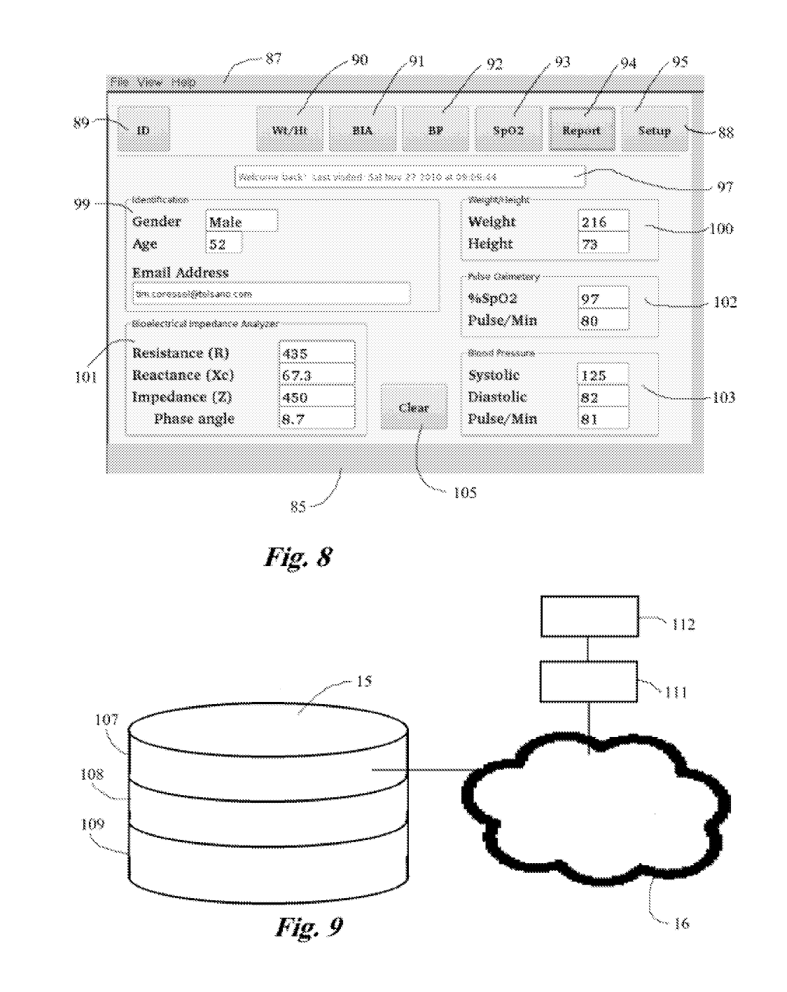

FIG. 8 shows an example of a graphical display 85 that the graphical user interface 82 may present to a user on the display 32 on the console 20 during a normal biometric data collection session. Along the top of the graphical display 85 is a menu bar 87 that provides dropdown “File”, “View” and “Help” menus. Below the menu bar 87 is a horizontal row of input buttons 88, including an identification button 89, a weight and height button 90, a bioelectrical impedance analysis system button 91, a blood pressure button 92, a pulse oximetry button 93, a report button 94, and a setup button 95. Below the input buttons 88 is a message field 97.

Below the message field 97, on the left side of the graphical display 85, is an identification field 99, and on the right side is a weight and height field 100. Below the identification field 99 is a bioelectrical impedance analysis system field 101. A pulse oximetry field 102 is located below the weight and height field 100, and below the pulse oximetry field 102 is a blood pressure field 103. A clear button 105 is located between the bioelectrical impedance analysis system field 101 and the blood pressure field 103.

A user initiates a biometric data collection session by providing that user's identification, such as an email address or other identifier. If the data collection station 14 is connected to the network management system 15, a message from the network management system 15, such as “Welcome back. Last visited Sat. Nov. 27, 2010 at 09.09.44” may be displayed in the message field 97. The user then selects the relevant input button 88, and manipulates the appropriate hardware. The data collection station 14 makes and records the selected biometric measurements, and displays the measurements in the appropriate field.

The data collection station 14 provides only temporary storage of biometric measurements. Biometric measurements can be printed and can be uploaded through the multi-media output interface 75 and the internet 16 and/or cellular system to the network management system 15. Referring to FIG. 9, the network management system 15 includes a communication manager 107, a content manager 108 and a data manager 109. The network management system 15 manages and controls the biometric data, the content sent to the data collection stations 14, the communication with the data collection stations 14, and operability and maintenance of the biometric measurement devices 17.

The network management system 15 has the ability to locate and manage each data collection station 14 that is connected to the internet 16 or cellular network. The communications manager 107 tracks, in real-time, the number of data collection stations 14 operating in the network, the current use of all data collection stations 14, the operability and usability of all data collection stations 14 and the operability and usability of the individual biometric measurement devices 17 integrated in each of the data collection stations 14.

The content manager 108 manages the content that is transmitted to the data collection stations 14 as well as mobile devices. The content can include advertising, coupons, and information.

The data manager 109 stores and retrieves the biometric data generated from all of the data collection stations 14. The data manager 109 can report the data as an overall aggregate data set and can sort by Zip Code, biometric measurement type (weight, height, blood pressure etc.), gender, age, and ethnicity. The data manager 109 can report the data to electronic health records or medical health records.

The network management system 15 has a secure website 111 that can be accessed by the user through a web capable device 112 such as a personal computer, a smart phone or any type of mobile device. A user can access the network management system 15 through the website 111 to track the user's biometrics such as weight, height, bioelectrical impedance analysis, body mass index, blood pressure, pulse oximetry and body temperature online. The website 111 can display the client's biometrics in an attractive graphically displayed representation.

Each user has an online account and profile. The profile includes a “community” portion where a user can designate other individuals that are allowed to access and view the user's biometric data. The network management system 15 can send real-time notices of when a set of measurements was taken and can also receive the actual content of the measured biometric data either by email or text message to the user and to individuals in the user's “community”. The network management system 15 can also send a message to remind the user as well as individuals in the user's “community” that the user has not taken a measurement in a prescribed period of time such as the last 30 days or last 24 hours. The network management system 15 combines a user's measured personal biometrics and compares the user's measured personal biometrics to guidelines such as, but not limited to, the guidelines defined by National Institute of Health for body mass index, weight, blood pressure, pulse oximetry and derive a single health score.

Although the present invention has been described with a certain degree of particularity, it is understood that the present disclosure has been made by way of example and that changes in details of structure may be made without departing from the spirit thereof.

What is claimed is:

1. A biometric healthcare data system comprising:
at least one portable data collection station having a base, a console, an elongated console support projecting upwardly from said base that supports said console above said base, and a plurality of biometric measurement devices connected to said console, and
a network management system in communication with said data collection station and having a communications manager that locates and manages said data collection station, a content manager that provides content to said data collection station and a data manager retrieves and stores data from said data collection station.

2. The system as set forth in claim 1 wherein said plurality of biometric measurement devices include a bioelectrical impedance analysis system, a weighing scale, and a height measuring device.

3. The system as set forth in claim 2 wherein said plurality of biometric measurement devices include a pulse oximetry sensor and a blood pressure meter.

4. The system as set forth in claim 2 wherein said bioelectrical impedance analysis system includes two spaced electrode sets of foot electrodes, two spaced electrode sets of hand electrodes, a multiplexer connected to said foot and hand electrodes, and a bioelectrical impedance analyzer connected to said multiplexer, with each said electrode set including a source electrode and a spaced detector electrode, said bioelectrical impedance analysis system measuring the resistance and the reactance for each combination of source electrode and detector electrode and generating resistance and reactance values for the right body, left body, lower body, upper body, right arm, left arm, right leg, left leg, right torso, left torso, transverse right arm to left leg, transverse left arm to right leg, and whole body of a user.

5. The system as set forth in claim 2 wherein each said electrode set of hand electrodes includes a block of non-conductive material, each said block having an upper surface and a spaced, oppositely facing lower surface, said detector electrode being a conductive rectangular plate on said upper surface and said source electrode being a conductive rectangular plate on said lower surface, said upper and lower surfaces being spaced a sufficient distance to position the fingertips of a hand on said source electrode and the thumb of said hand on said detector electrode, and to prevent the palm of said hand from contacting said hand electrodes,
whereby repeatability of measurements is improved.

6. The system as set forth in claim 5 wherein:
said console has a flattened box shape with an upper face, a spaced lower face, spaced left and right side walls that extend from said upper face to said lower face, and spaced front and back walls that each extend from said left side wall to said right side wall, said console being tilted such that said upper face faces upwardly and forwardly, and
one said block is mounted on said left side wall and one said block is mounted on said right side wall of said console

7. The system as set forth in claim 1 wherein said console is removably attached to said console support and said console support is removably attached to said base,
whereby said data collection station can be disassembled for transport.

8. The system as set forth in claim 1 wherein said network management system connects to said data collection station through at least one of the internet and the cellular network.

9. The system as set forth in claim 1 wherein said network management system has a secure website accessible through a web capable device.

10. The system as set forth in claim 9 wherein said network management system includes an online account and a profile for each user, said profile having a community portion designating other individuals that are allowed to access and view biometric data of said user.

11. The system as set forth in claim 10 wherein said network management system sends notices, alerts and reminders to said user and to individuals in said community portion.

12. The system as set forth in claim 1 wherein said communications manager tracks the operability and usability of said data collection station and the operability and usability each of said plurality of biometric measurement devices.

13. A biometric healthcare data system comprising:
at least one portable data collection station having a base, a console, an elongated console support projecting upwardly from said base that supports said console above said base, and a bioelectrical impedance analysis system, a weighing scale, a height measuring device, a pulse oximetry sensor and a blood pressure meter connected to said console, said bioelectrical impedance analysis system including two spaced electrode sets of foot electrodes, two spaced electrode sets of hand electrodes, a multiplexer connected to said foot and hand electrodes, and a bioelectrical impedance analyzer connected to said multiplexer, each said electrode set including a source electrode and a spaced detector electrode, said bioelectrical impedance analysis system measuring the resistance and the reactance for each combination of source electrode and detector electrode and generating resistance and reactance values for the right body, left body, lower body, upper body, right arm, left arm, right leg, left leg, right torso, left torso, transverse right arm to left leg, transverse left arm to right leg, and whole body of a user, each said electrode set of hand electrodes including a block of non-conductive material, each said block having an upper surface and a spaced, oppositely facing lower surface, said detector electrode being a conductive rectangular plate on said upper surface and said source electrode being a conductive rectangular plate on said lower surface, said upper and lower surfaces being spaced a sufficient distance to position the fingertips of a hand on said source electrode and the thumb of said hand on said detector electrode, and to prevent the palm of said hand from contacting said hand electrodes, and
a network management system in communication with said data collection station and having a communications manager that locates and manages said data collection station, a content manager that provides content to said data collection station and a data manager retrieves and stores data from said data collection station.

14. A portable data collection station for a biometric healthcare data system, comprising:
a base,
a console,
an elongated console support projecting upwardly from said base that supports said console above said base,
a portable gateway in said console, and
a plurality of biometric measurement devices connected to said gateway including a bioelectrical impedance analysis system, a weighing scale, a height measuring device, a pulse oximetry sensor and a blood pressure meter connected to said console.

15. The data collection station as set forth in claim 14 wherein said bioelectrical impedance analysis system includes two spaced electrode sets of foot electrodes, two spaced electrode sets of hand electrodes, a multiplexer connected to said foot and hand electrodes, and a bioelectrical impedance analyzer connected to said multiplexer, with each said electrode set including a source electrode and a spaced detector electrode, said bioelectrical impedance analysis system measuring the resistance and the reactance for each combination of source electrode and detector electrode and generating resistance and reactance values for the right body, left body, lower body, upper body, right arm, left arm, right leg, left leg, right torso, left torso, transverse right arm to left leg, transverse left arm to right leg, and whole body of a user

16. The data collection station as set forth in claim 15 wherein said each said electrode set of hand electrodes includes a block of non-conductive material, each said block having an upper surface and a spaced, oppositely facing lower surface, said detector electrode being a conductive rectangular plate on said upper surface and said source electrode being a conductive rectangular plate on said lower surface, said upper and lower surfaces being spaced a sufficient distance to position the fingertips of a hand on said source electrode and the thumb of said hand on said detector electrode, and to prevent the palm of said hand from contacting said hand electrodes,
whereby repeatability of measurements is improved.

17. The data collection station as set forth in claim 16 wherein:
said console has a flattened box shape with an upper face, a spaced lower face, spaced left and right side walls that extend from said upper face to said lower face, and spaced front and back walls that each extend from said left side wall to said right side wall, said console being tilted such that said upper face faces upwardly and forwardly, and
one said block is mounted on said left side wall and one said block is mounted on said right side wall of said console.

18. The data collection station as set forth in claim 14 wherein said console is removably attached to said console support and said console support is removably attached to said base,
whereby said data collection station can be disassembled for transport.

19. The data collection station as set forth in claim 14 wherein said gateway includes a multi-media output interface that connects said gateway through at least one of to the internet and the cellular network to a network management system for uploading biometric healthcare data generated by said biometric measurement devices.

20. The data collection station as set forth in claim 19 wherein said gateway includes multi-media input interface connected to said plurality of biometric measurement devices, a protocol converter connected to said multi-media input interface, a central processing unit connected to said a protocol converter, a user interface controller, for connection of user interface devices, connected to said central processing unit, and a peripheral interface adapter, for connection of peripheral devices, connected to said central processing unit.
